(19) United States
(12) Patent Application Publication
(10) Pub. No.: US 2010/0274116 A1
(43) Pub. Date: Oct. 28, 2010

(54) COMPUTER-SUPPORTED MEDICAL IMAGE ACQUISITION AND/OR ASSESSMENT

(76) Inventors: Thomas Blum, Neunkirchen A. Br (DE); Roland Faber, Uttenreuth (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/766,201

(22) Filed: Apr. 23, 2010

(30) Foreign Application Priority Data
Apr. 24, 2009 (DE) 10 2009 018 875.4

(51) Int. Cl. A61B 5/05; G06F 17/30

(52) U.S. Cl. 600/407; 707/722; 707/E17.019; 707/705; 707/802; 707/769

(57) ABSTRACT

In a method for supported recording and/or analysis, in particular assessment, of images which are to be and/or have been recorded with an imaging device using a database containing medical information, in particular a medical atlas, in the imaging device and/or in a computer, following a command from a user and/or through the occurrence of a triggering event, dependent existing image related data, at least one image related information item is selected from the database automatically, and the at least one, image related information item is displayed.

### BACKGROUND OF THE INVENTION

1. Field of the Invention

The present invention concerns a method for supported recording (acquisition) and/or analysis of, in particular the appraisal of, images which are to be and/or have been recorded with an imaging device, as well as an imaging device for this purpose and a suitable storage medium specifically for use by a referring physician.

2. Description of the Prior Art

The acquisition and particularly the assessment of medical images is a complex field of expertise which, aside from a certain amount of experience, also requires a fundamental medical, particularly anatomical, knowledge. In medical imaging, a large number of high-resolution, in part, highly detailed, images of human anatomy have been produced. Within limits, due to the large number of medical imaging processes and the resulting differences in their results, a clear recognition of the anatomical structures, or respectively, the distinction between physiological and pathological changes is often quite difficult and can only be accomplished by highly trained medical experts. These problems affect all of the professional fields involved in the acquisition and assessment processes of said images.

For this reason, the medical technician assistant, the individual normally responsible for operating the imaging modality, must obtain the best possible image quality in the framework of the prescribed examination, and make a comprehensive acquisition of the region being examined. Physiological and pathological changes in the human body may lead to a need for adapting the examination protocol or the imaging parameter in order to achieve this aim. In addition it is necessary that the anatomical structure be clearly recognizable.

The evaluating physician, particularly the radiologist, must be able to distinguish pathologically altered structures from physiological changes or variations between different individuals, and develop a medical assessment.

As a result, the referring physician, for example a clinic physician or a general practitioner, increasingly receives additional images for supplementary clarification or for conferring with the patient along with the medical report from the reporting physician, i.e. the radiologist, and must therefore be capable of interpreting and explaining these images.

In order to enable access to the necessary anatomical information, many medical imaging workstations and particularly image assessment workstations have medical atlases available in book form. The search for appropriate presentation examples and further information thereby is however time-consuming, and when the diagnostic workstation is dimly lit, can only be carried out with difficulty, if at all.

Furthermore, segmentation techniques are known which provide assistance in the recognition of anatomical structures, but are of little use in distinguishing physiological and pathological changes. These segmentation techniques are in part quite labor intensive, requiring high image quality and computing power, and are not always available in medical centers.

In particular, the referring physician usually has only the reported image material and a simple image viewer available.

### SUMMARY OF THE INVENTION

An object of the present invention is to improve the means of making the actual medical, particularly anatomical, data of the examination available in a simple and immediate manner, and thereby to improve the quality of the recorded images and assessments.

This object is achieved in accordance with the invention by a method for computer-supported acquisition and/or analysis, particularly reporting, of images which are to be and/or have been recorded by an imaging device through the use of a database of medical information, in particular a medical atlas, in the imaging device and/or a computer wherein following a predetermined command by an operator and/or the occurrence of a triggering event, dependent on existing image related data, at least one image related information item is automatically selected from the database, and this at least one image related information item is displayed.

A context related support from a database having medical information, ideally from a medical atlas that can be queried by the user, and the selection is made from immediately familiar image related data that have been entered, for example, through an imaging device, downloaded from an information system, or simply included with the image. For this purpose, the information item that is to actually be displayed, if enough image related data are available, is provided and displayed fully automatically based on the image related data. It is also possible for the image related data to be displayed to be determined and displayed based on additional image related data entered by a user, which may be accomplished for example through a user-friendly search template. Nevertheless, already existing image related data are used in the last, semi-automatic case in order to make a pre-selection or partial selection from the database.

As a result, the method of the invention may be used to support the acquisition of images, but also, particularly, to support the assessment. Anatomical information is also often necessary in order to correctly adjust the imaging parameters of an imaging device, or in order to correctly interpret and use previously recorded orientation images such as localizers. Consequently, the method of the invention may also be used advantageously and beneficially to support the acquisition of images.

The method provides access to a source of medical information, the database, form an imaging, assessing, or image viewing workstation, whereby image related information is used to make, at least a semi-automatically, a selection of medical background information. This has numerous advantages. The image quality is improved in the framework of acquisition the image through the support provided in creating the images. In addition, the assessment quality is also improved in the framework of the assessment as targeted information for assessments to be carried out can be supplied. Not least of all, in all of the areas of application of the process of the invention, time is saved by eliminating time-consuming searches for background information. A referring physician less experienced in the practice of interpreting images can furthermore be made aware of specific supplementary medical information. Furthermore, the method of the invention may also be used in the framework of instruction or training to particular advantage, for example when new medical technician assistants or physicians in the early stages of their careers are confronted with the problems involved in image acquisition or assessment.

The concept of image related data in the framework of the present process encompasses a large range, and ultimately includes all of the specialized identifying information involved in an examination. Consequently, image related data may refer to patient related data, particularly the age and/or the sex and/or the weight of the patient, and/or acquisition parameters and/or the imaging region and/or data indicating a possible diagnosis and/or data derived directly from the image itself, particularly segmenting relevant data. Patient related data is usually the first information provided to an imaging device, either through entering said by a user in the course of registering the patient, or alternatively through a referral produced and transmitted through an information system. Aside from the general demoscopic data, such as age, sex and weight, often the data of an electronic patient file is available, which may give information regarding specific physiological information and can also be taken into consideration advantageously in selecting information from the database. In addition, it is also frequently clear from existing image related data as to what type of examination is to be carried out, indicating which region of the body, particularly which organ, is to be examined, and what possible diagnoses are to result from the examination. Finally, data from the image may also be used, as a matter of course, for example when a segmenting of one or more organs is to be executed, or for similar means. In this manner it can be anticipated that with an image being displayed, in which an organ is segmented and selected, by executing the command, or as a result of a triggering event, data for this organ is made immediately and fully automatically available.

Appropriately, at least a portion of the image related data may be provided by referral from an information system, in particular from a hospital information system or a radiology information system, and/or from an electronic patient file and/or a header on the image containing metadata, particularly a DICOM-header. While the information systems and the electronic patient files have been discussed above, it is advantageous for the assessment, but also for the field of the imaging, when a header is already created before the acquisition of image data, if a header containing image related data which is already in the datum of the image data, can be implemented. This type of header is typical, particularly in the DICOM-format, such that aside from patient related data to be found there, imaging parameters such as the modality used, the application of a contrasting agent etc. can be stored and made available for the assessment as well as for the purpose of selecting information from the medical database.

The data to be displayed may be in the form of automatic data and/or if applicable, additional images of a recorded region with supplementary information and/or an organ which is to be imaged and/or text information and/or determined imaging parameters, in particular an existing image created with said imaging parameters, and/or information for distinguishing between pathological and physiological changes. Here as well, numerous possibilities are apparent, as to how the medical data may be beneficial in the framework of the process of the invention in the form of information to be displayed can be processed. In this manner, it is possible for the assessment that, for example, an anatomic diagram with a standardized breakdown of an organ and additional information could be displayed. In reference to specific questions, text information and/or illustrations may be used for distinguishing between physiological changes and pathological changes. Of particular benefit in the framework of the invention are also comparable, previously recorded images with supplementary information, such as, for example, images in which specific anatomic characteristics can be identified through highlighting, reference marks or similar items. In the field of imaging, it is particularly advantageous when images—also when, if applicable, images supplemented with further information—are displayed, particularly with respective imaging parameters, that reproduce how the aim of the examination may be obtained using certain imaging parameters. As an example, for an ultrasound apparatus a specific placement of the ultrasound head may be described and illustrated, in particular in relation to the anatomy of the patient, as well as an image recorded from this ultrasound head placement. Clearly, any number of possibilities is conceivable.

As mentioned above, the selection and displaying of the information can be initiated through a command by the user. In addition, one possibility for the user to trigger a command would be to use a specific key on a keyboard and/or to make a selection from a menu. As an example, a hotkey could be used, e.g. the F1, and/or a menu, which could also allow the help function to be opened. In this manner, the user can access the appropriate information from the database comfortably, through a single touch of a button. Alternatively, or additionally, it is also conceivable that the selection and displaying of information could be achieved through a triggering event. This would ultimately result in an automatic display of information when, for example, a segmenting is completed, or something of a similar manner. Incidentally, it would also be possible—in order to make the selection easier for less experienced user, but no longer displaying this for more experienced users—to make use of user profiles which regulate the display of information through triggering events. As an example, it would be possible to have a workstation with numerous display monitors in which one monitor is used to display image related information.

In other versions of the invention, it would be possible that after displaying the information, further information could be interactively selected from the database and displayed. For example, certain written terms could be displayed as links through which further information can be called up, and it is also conceivable that, for example, by clicking on specific regions in an image, further information pertaining to said region could also be called up. In this manner, the user is able to obtain additional information pertaining to the displayed information in a simple fashion.

Alternatively, the database may be accessed through a network, in particular through the internet, and/or that the database is stored locally with the data, in particular on a storage medium that can be provided to the referring physician. Thus, an online available database is conceivable, which is stored for example in a network, such as the Internet, or alternatively, an intranet of a medical institute such as a clinic. In this case, the data bank, and thereby the medical information, can be accessed from numerous workstations. In this manner, everybody accessing the data bank receives the same or comparable information. Alternatively, it would be possible for said only to be available locally, in other words, stored in the imaging device or the computer, for example. It can also be particularly advantageous for the database to be saved together with the data on a storage medium such as a CD or DVD that is to be provided to the referring physician. A programming resource, such as an image viewer can also be added to this, which allows access to the database described herein. This enables, in a simple manner, the referring physician to access the medical information in the database, which he would normally not have access to, and thereby enabling the referring physician to make a better interpretation of the image. This type of database containing medical information can normally be saved on a CD or DVD, such that only the actual image data needs to be added in order to provide the referring physician with an ideal tool for further processing and/or interpretation of the image.

In a particularly advantageous version of the process of the invention, it would be possible that a specific medical facility, in particular a clinic, is designated for use with this configured database. In this manner, certain standards, such as clinic standards, could be circulated, such as the specific terminology for certain anatomic characteristics or disease patterns, while at the same time it would also be possible to align the assessment practices and references of various physicians, so that the assessment results of different physicians correspond. A medical facility of this type can, for example, also be a central clinic with numerous subsidiary clinics, a medical practice or an association of numerous medical practices, etc.

Accordingly, the information to be displayed would be displayed together with the image. The appraising physician can, in this manner, view the data together with the image, and is thereby able, for example, to directly compare the distinction between physiological and pathological changes. As an example, the display of the information from the data bank can be presented such that it occupies an area which is not occupied by the image.

As noted, the information to be displayed may also contain imaging parameters, in particular if it contains a sample image or similar item. In this case, it would be possible that the imaging parameter in the information to be displayed would be used to control the imaging device. For example, a control element may be incorporated in the information presentation, which would result in the acquisition of the imaging parameters by the imaging device, whereupon the imaging device would then be controlled by said imaging parameters. In this manner, the operating of the imaging device would be further simplified.

Analogously, the information to be displayed, particularly if it contains a previously analyzed sample image or similar item, also contains imaging parameters. Accordingly, the evaluation parameters contained in the information to be displayed can then be used for analyzing the image. In this manner, the analysis procedure for analyzing the image, which contains physical-technical data, can be further improved and the comparability with the information to be displayed thereby improved. As an example, it would be possible that a segmenting carried out in the information to be displayed could be used with the image.

The method of the invention is suited for practically all known types of medical imaging processes, such as magnetic resonance imaging, X-ray images, CT-images, ultrasound images, intracorporeal or extracorporeal images.

In addition to the method, the invention encompasses an imaging device, specifically an ultrasonic device, having a control device specifically designed for executing the process of the invention, a display device and an input device, whereby in the framework of the acquisition and/or analysis of an image the control device is designed such that at least one image related datum from a database, stored either in the control device or accessible through a network having medical data based on existing image related data, may be selected using a predetermined command from a user through the input device, and to display said, or at least one, image related data on the display device. The method of the invention can, therefore, be used directly with an imaging device which is programmed for said, such that the data from the database in a contextual relation to a user, whether it is during the acquisition or the analysis of images, can be made available. In this manner, the time expenditure for more complex acquisition of the information is eliminated as well with the process of the invention, and an imaging device of this sort can be used to particular benefit for instruction and training purposes with new users. Furthermore, the above statements regarding the process of the invention are in all cases analogous to the imaging device of the invention.

Naturally any diagnostic workstation or image viewing workstation with a computer can be configured so as to be capable of executing the method by installing appropriate programming resources on the computer.

The present invention also encompasses a storage medium in which an image, containing, in particular, image related data and a programming resource is stored. The programming resource is designed for executing the method and all embodiments thereof described above. A storage medium of this sort can beneficially, as already explained, be provided to a referring physician, who would then have access to the medical information stored in the database, which would be displayed in a contextually sensitive manner. For this a particular benefit could be derived by saving the database on the storage medium, although is it basically also conceivable that, in the case where the physician has access to a network, particularly the internet, the programming resource is designed such that the database can be accessed through the network. The programming resource may also, particularly advantageously, take the form of an image viewing program, in order that the referring physician, when he has received the storage medium, need only install the program in order to view the, at least one, image(s) and simultaneously have the possibility, by initiating a command, of calling up and examining image related information from the database.

### BRIEF DESCRIPTION OF THE DRAWINGS

FIG. 1 is a flowchart of a first embodiment of the method in accordance with the invention.

FIG. 2 is a flowchart of a second embodiment of the method in accordance with the invention.

FIG. 3 shows an imaging device in accordance with the invention.

FIG. 4 shows a workstation designed to execute the process in accordance with the invention.

FIG. 5 is a storage medium in accordance with the invention.

### DESCRIPTION OF THE PREFERRED EMBODIMENTS

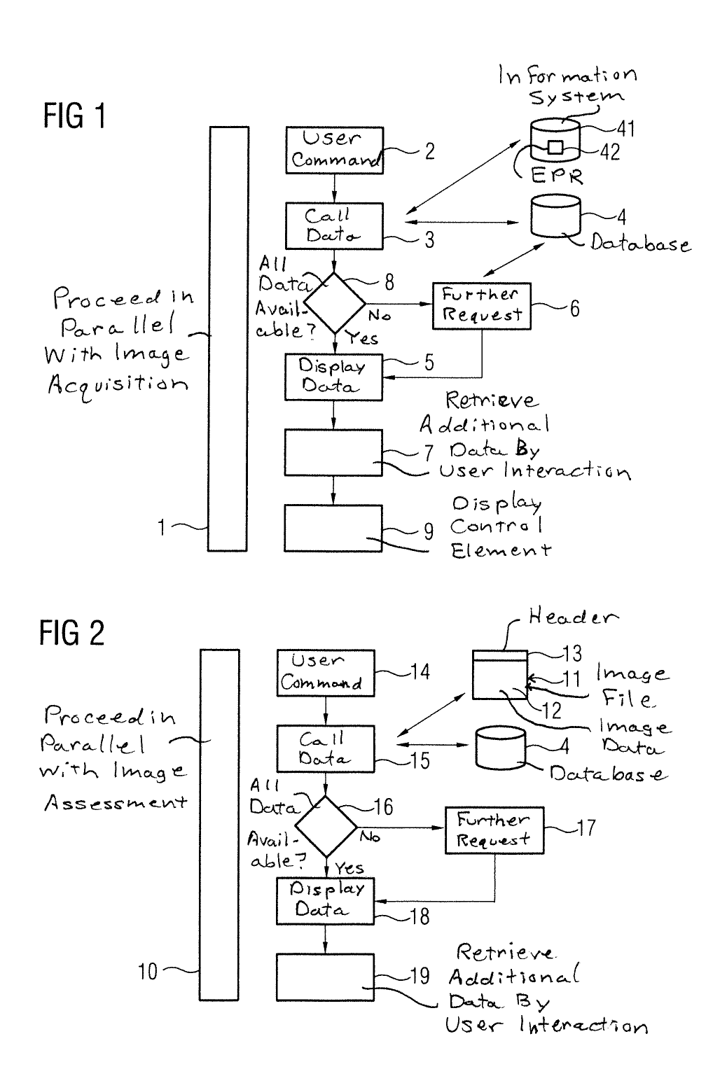

FIG. 1 shows basic components and steps of a first embodiment of the process in accordance with the invention. This is primarily an embodiment executed by an imaging device during the acquisition of images by the imaging device. Step 1 indicates that the first embodiment functions in parallel to the indicated imaging process. In a second step 2, a user, in this case, for example a medical technician assistant in charge of making the acquisition, initiates a predetermined command, for example by using a specific key on a keyboard, particularly the F1 key, or making a selection from a menu. Subsequently, in step 3 it is determined which image related data are needed, in order to automatically call up at least one image related item from a database 4 containing medical information based on the data, ideally from a medical atlas, which may be used in context to support the user.

Such existing image related data may be data regarding the patient, such as age, sex and weight, or indicating the region to be imaged and a possible diagnosis. Imaging parameters, already set by a user, or the basic information of the imaging device may naturally also be taken into consideration. The patient and examination related data may originate in, for example, a request from an information system 41, such as a radiology information system or a hospital information system, which may also contain electronic patient records (EPR) 42. Image related data of this type may also be retrieved directly from the information system 41, and the data may also, at least in part, be entered manually by the user.

Two possibilities are to be considered, which are represented by step 8. Should it be the case that sufficient image related data is available in order to reasonably limit the displayed image related information, then the image related information to be displayed may be displayed in step 5 on a monitor. Should it be the case that insufficient image related information is available, then the user can, in step 6, reference further information through a search template. A search template of this sort should be relatively user-friendly, such as an abstracted anatomic representation, from which a desired organ or similar item may be selected. If the image related data is then sufficient to reasonably select image related data which is to be displayed from the database 4, it will then be displayed in step 5.

In a step 7, the possibility exists for the user to interactively retrieve additional information, based on the displayed image related data, from the database 4, such as detailed anatomical information by marking a specific region or link in regard to specific pathologic or physiologic changes which are indicated in writing. Any number of possibilities is conceivable here.

The information from the database 4 shown in step 5 is for the most part anatomic information which assists the user thereby in adjusting the imaging parameters for said particular examination, in this case said particular imaging. For this purpose, assistance may be provided in, for example, interpreting an existing acquisition of an orientation image, specifically a localizer, and thereby, for example, distinguishing physiological or pathological changes and appropriately adjusting the imaging parameters. The displayed image related information may already contain some imaging parameters, such that a control element may be implemented through which, as is indicated in step 9, the imaging parameters may be used directly for the purpose of controlling the imaging device, whereby they will accordingly be implemented.

The database 4 used here may be stored locally, for example in the imaging device itself, although it is also possible to access said through a network. It should be noted at this point that it is the case for essentially all design versions that the database 4 may be specifically configured for a specific medical facility, such as a clinic, in that, for example, reference standards and guidelines which are to be observed or similar items are integrated with the medical information.

FIG. 2 shows a possible second embodiment in accordance with the invention, during the analysis, indicated by step 10, of an existing image (or numerous images), which for the most part functions in a similar manner to the first embodiment. In this case, the image already exists as a file 11, which aside from the image data 12, also contains a header 13, ideally a DICOM-header, in which relevant image related data are saved. These may be similar data to those already specified, including patient data, the imaging modality, various imaging parameters, or also data for an existing segmenting or regarding the application of a contrasting agent.

Should a command be executed by the assessor in his analysis workstation, as has already been described in the first embodiment, step 14, then in step 15, taking into consideration the available image related data in the header 13 which is in turn a selection of image related data from the medical information contained in the database 4, which in turn may be locally available, in particular if it is the case that it refers to an analysis by a referring physician, may also be on an included storage medium or may be accessed through a network, such as the internet.

Subsequently it is determined in step 16 if the present image related data are sufficient or if further image related information must be retrieved through a search template in step 17, in order that in step 18 the final selection of image related information may be displayed. Here as well, there is the possibility in a step 19 to interactively retrieve additional information from the database 4. In this manner, the analysis is supported, particularly when comparison images, anatomic information etc. can be displayed as image related information. It is also possible here that—for example by activating a control element—assessment parameters for assessing the image contained in the information can be used.

It is emphasized that the display in step 18 is shown simultaneously with the image to particular advantage, thus enabling a direct comparison.

Even though the design example of FIG. 1 refers to the imaging process and the design example of FIG. 2 refers to the analysis, it should however be emphasized that the process of the invention may be implemented in both functions.

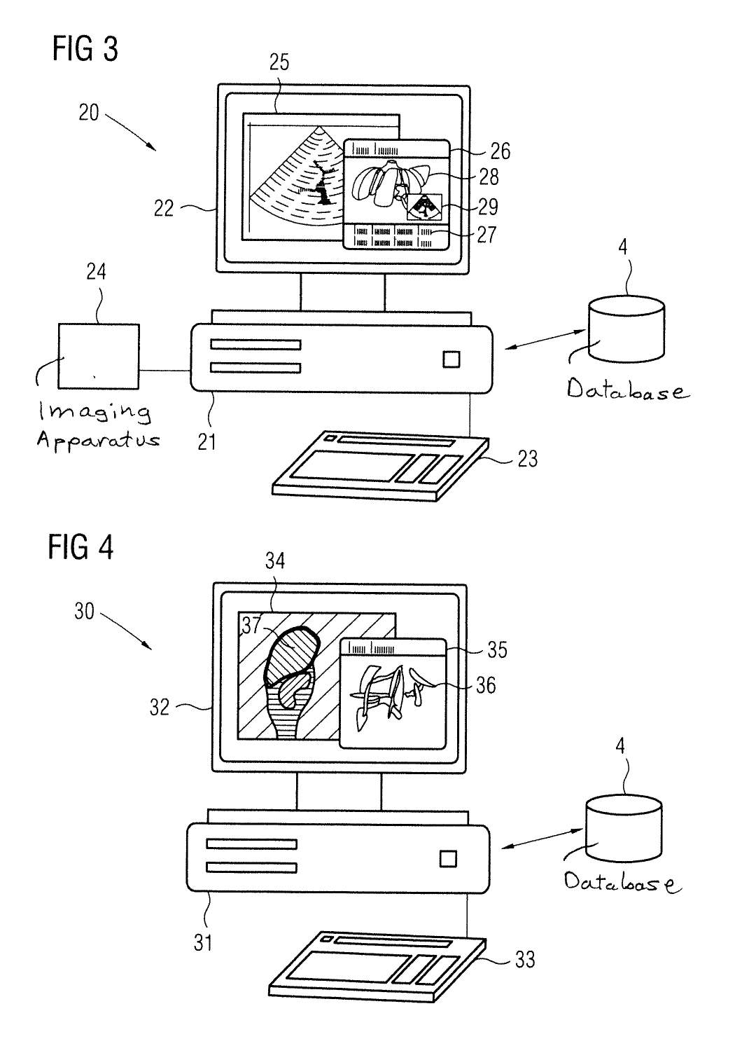

FIG. 3 shows essential components for the present invention of an imaging device 20 in accordance with the invention, primarily an ultrasound device; specifically a control device 21, a display device 22 and an input device 23, whereby the actual imaging apparatus 24 contains, for example, the ultrasonic head, which is merely indicated here. The control device 21 thereby is designed for executing the process of the invention, particularly the process according to FIG. 1, i.e. by executing a specific command through the input device 23, image related information can be retrieved from the database 4 and thereby displayed.

FIG. 3 also shows a possible presentation on the display device 22, which contains an actual live image 25 as well as the displayed image related information 26. The image related information available here is text information 27, an anatomical presentation 28 of the organ to be examined with a classification, a standardized description and various anatomic characteristics as well as an example image 29 with relevant specific parameters. Said information enables the user, due to the easily accessible anatomic, or respectively, medical background knowledge, the imaging to be carried out such that the aim of the examination can be obtained with high image quality.

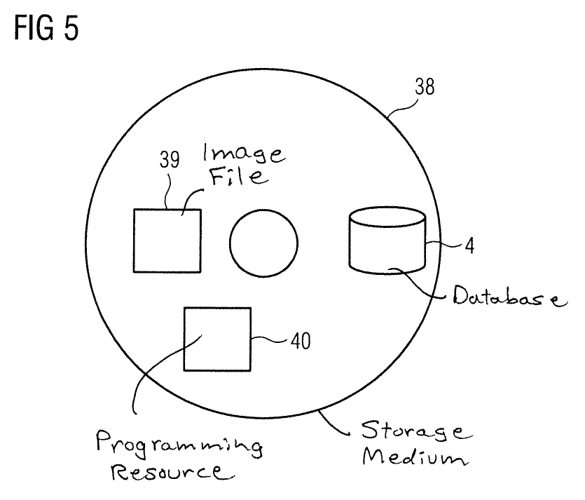

FIG. 4 shows an analysis workstation 30 with a computer 31, a display monitor 32 and an input device 33. The computer 31 is programmed to execute the process of the invention, in particular in accordance with FIG. 2. The database 4 is stored on the control device 31, or said control device 31 has access to the database 4 through a network. Once again, an exemplary presentation is shown on the display monitor 32. The presentation contains the image being analyzed 34, which is displayed simultaneously with the image related medical information 35 selected from the database 4, which in this case is composed of basic anatomical information 36 of a segmented organ 37 in the image 34. In this case incidentally, the selection of the information from the database 4 to be shown could be carried out automatically, in that the organ 37 was selected and thereby all essential information is accessed thus allowing for locating and presenting the correct information in context. The rest of the necessary image related information may be found in the header 13.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for computer-supported acquisition or assessment of a medical image, comprising the steps of:
providing a computer with an initial image-related entry for a medical image, selected from the group consisting of an entry at least partially designating a medical image to be acquired with a medical imaging apparatus, and an entry designating an already-acquired medical image;
following said initial image-related entry, waiting in said computer for an occurrence selected from the group consisting of a user command and a triggering event;
in response to said occurrence, automatically, in said computer, accessing a database that contains image-related information and, dependent on said initial image-related entry, retrieving at least some of said image-related information from said database, as retrieved image-related information; and
at a display in communication with said computer, visually displaying said retrieved image-related information.

2. A method as claimed in claim 1 comprising automatically displaying all of said retrieved image-related data at said display.

3. A method as claimed in claim 1 comprising modifying the retrieved image-related information according to at least one modifying entry entered into said computer, to obtain modified image-related information, and displaying said modified image-related information at said display.

4. A method as claimed in claim 1 wherein said medical image is a medical image of a patient, and comprising storing image-related information in said database selected from the group consisting of the age of the patient, the sex of the patient, the weight of the patient, imaging parameters for acquisition of said medical image, a region of the patient to be shown in said medical image, data relevant for a diagnosis of the patient, data relating directly to contents of said medical image, and data defining segmenting of said medical image.

5. A method as claimed in claim 1 wherein said database is selected from the group consisting of a hospital information system, a radiology information system an electronic patient file, a metadata-containing header for said medical image, a DICOM header for said medical image, and comprising retrieving said retrieved medical image information from said database by initiating a request to said database from said computer.

6. A method as claimed in claim 1 comprising displaying said image-related information in a form selected from the group consisting of anatomical information, an image of a region of a subject supplemented with additional information, an image of an organ of a subject, text information, imaging parameters, and information distinguishing between pathological and physiological changes.

7. A method as claimed in claim 1 wherein said occurrence is a user command and selecting said user command from the group consisting of a command designated by activation of a specific keyboard key of a keyboard in communication with said computer, and a command designated by a selection from a menu displayed at said display.

8. A method as claimed in claim 1 comprising, after displaying the retrieved image-related information at said display, retrieving additional image-related information from said database via said computer.

9. A method as claimed in claim 1 comprising physically locating said database at a location remote from said computer, and accessing said database from said computer via a network.

10. A method as claimed in claim 1 comprising storing said database in a storage medium and loading said storage medium into said computer.

11. A method as claimed in claim 1 comprising configuring said database to conform to requirements of a predetermined medical facility.

12. A method as claimed in claim 1 comprising displaying said retrieved image-related information at said display together with said medical image.

13. A method as claimed in claim 1 wherein said medical image is a medical image to be acquired with a medical imaging apparatus, and comprising storing and retrieving image-related information defining parameters for acquiring said medical image with said medical imaging apparatus.

14. A method as claimed in claim 1 wherein said medical image is an already-acquired medical image, and comprising storing and retrieving image-related information for medically assessing said image to obtain a medical diagnosis.

15. A medical imaging device for computer-supported acquisition or assessment of a medical image, comprising:
a computerized processor:
said processor having an input through which said processor is supplied with an initial image-related entry for a medical image, selected from the group consisting of an entry at least partially designating a medical image to be acquired with a medical imaging apparatus, and an entry designating an already-acquired medical image;
said processor being configured to wait, following said initial image-related entry, for an occurrence selected from the group consisting of a user command and a triggering event;
a database containing image-related information;
in response to said occurrence, said processor being configured to access said database and, dependent on said initial image-related entry, retrieving at least some of said image-related information from said database, as retrieved image-related information; and
a display in communication with said processor, said processor being configured to visually display said retrieved image-related information at said display.

16. A non-transitory, computer-readable storage medium encoded with programming instructions, said storage medium being loaded into a computer and said programming instructions causing said computer to:
receive an initial image-related entry for a medical image, selected from the group consisting of an entry at least partially designating a medical image to be acquired with a medical imaging apparatus, and an entry designating an already-acquired medical image;
following said initial image-related entry, wait for an occurrence selected from the group consisting of a user command and a triggering event;
in response to said occurrence, automatically access a database that contains image-related information and, dependent on said initial image-related entry, retrieve at least some of said image-related information from said database, as retrieved image-related information; and
at a display in communication with said computer, visually display said retrieved image-related information.
